(19) United States
(12) Patent Application Publication
(10) Pub. No.: US 2025/0283143 A1
(43) Pub. Date: Sep. 11, 2025

(54) Smart chemo-enzymatic color change label and expiration date of food, pharmaceutical, and biological products

(71) Applicants: Mohammadhossein Sharifnia, Tehran (IR); Mohammadhossein Sharifnia, Tehran (IR)

(72) Inventors: Mohammadhossein Sharifnia, Tehran (IR); Mohammadmahdi Sharifnia, Tehran (IR)

(73) Assignee: Zahra Beheshti Marnani, Tehran (IR)

(21) Appl. No.: 18/709,066

(22) PCT Filed: Jan. 3, 2022
(86) PCT No.: PCT/IB2022/050022

§ 371 Date: May 10, 2024

(51) Int. Cl. C12Q 1/26

(52) U.S. Cl. CPC C12Q 1/26

(57) ABSTRACT

The invention is directed to an intelligent label designed for predicting, detecting, and monitoring the expiration date of sensitive products. The label aims to identify the realistic shelf life of the expiration date through its unique components and properties. The intelligent label consists of a nitrocellulose layer with specific dimensions serving as the foundation, a nitrate reductase enzyme, and a PAM monomeric derivative polymer with dual properties of temperature and moisture sensitivity, making it hydro-thermo-sensitive. This label exhibits dual active diagnostic and predictive behavior. It undergoes appearance, consistency, and color changes by enzymatic activity in the polymer structure to predict, estimate, and recognize expiration dates and the realistic product life, particularly sensitive to climate conditions. A temperature and time-sensitive chemozyme method is employed to study the effects of temperature and climate changes in food spoilage, pharmacological components degradation, and the protein and glycoprotein structures deformation of biological and medicinal products.

### FIELD OF THE INVENTION

The background of the invention is in the field of Medicinal preparations characterized by the non-active ingredients used, e.g. carriers or inert additives; Targeting or modifying agents chemically bound to the active ingredient (A61K 47/00)—Medicinal preparations containing organic active ingredients (A61K 31/00)—using chemical indicators (GOIN 31/22)—Forms or constructions (G09F 3/02)

### BACKGROUND OF THE INVENTION

The KR101209266B1 patent with the title of “Biodegradable and thermosensitive poly(phosphazene)-superparamagnetic nano-particle complex, preparation method and use thereof” provides ferrite superparamagnetic nanoparticles of iron oxide (Fe₃O₄, Magnetite), which are T-₂ and T₂*-enhanced magnetic resonance imaging contrast materials, and sol-gel according to temperature change.

1) phosphazene-type ‘bonded’ magnetic polymers produced through hydrophobic interaction with biodegradable and temperature-sensitive phosphazene-based polymers with functional groups and/or hydrophilic superphase with the phosphazene-based polymers; 2) phosphazene-based ‘mixed’ magnetic polymers produced through simple physical mixing with magnetic nanoparticles, and 3) methods of making these materials, use as biomaterials, bioactive mass transfer materials and hyperthermia biomaterials It relates to the use as a material. By producing and synthesizing nanoparticle-scale supramagnetic polymers, it has been studied and claimed that it is based on ferric and ferric ions in iron oxides, whose therapeutic and drug delivery effects have been claimed.

The U.S. Pat. No. 9,969,819B2 patent with the title of “Pressure sensitive adhesive including a 1,1-disubstituted alkene compound” has claimed that it is possible to polymerize 1,1-disubstituted alkene compounds in a solution (for example using one or more solvents). Polymerization of 1,1-disubstituted alkene compounds in a solution provides opportunities to better control the polymerization compared with bulk polymerization.

The solution polymerization techniques can be employed for preparing homopolymers, copolymers (e.g., random copolymers), and block copolymers. Alkene monomeric derivatives and compounds with two substitutions in alkene regions 1 and 1 have been investigated to produce different polymers that are sensitive to pressure changes. Applications of these polymer derivatives in the field of paint production, protective layer and masking of surfaces with adhesive properties have been mentioned.

In the U.S. Pat. No. 9,937,254B2 patent with the title of “Water-soluble supramolecular complexes” Water-soluble supramolecular complexes have been claimed that formed from a water-soluble block copolymer and at least one associative gelling adjuvant. The copolymer includes at least two blocks of polyethylene oxide and at least one block of polypropylene oxide.

The adjuvant has a water solubility less than 0.5 g/100 ml at 20° C. When combined with water, the complexes form a transparent reversely thermo-reversible hydrogel or solution that may be repeatedly hydrated and dehydrated. The hydrogel exhibits improved gelling efficiency and enhanced solubility and/or stability for sparely soluble and insoluble pharmaceutical agents.

The complexes are useful in a variety of pharmaceutical and cosmetic products and applications and may be combined with an effective amount of a cosmetic, medicament, or diagnostic in a solid dosage form. The synthesis and production of hydrogel polymers with sensitivity to inverse temperature changes and their reversible effects on the polymer have been studied and the drug delivery properties of these derivatives have been claimed to improve skin, eye, vaginal, and anal diseases.

In the U.S. Pat. No. 5,709,472A patent with the title of “Time-temperature indicator device and method of manufacture” a time-temperature indicator label has been claimed for measuring the length of time to which a product has been exposed to a temperature above a pre-determined temperature is provided.

The period of time of exposure is integrated with the temperature to which the indicator is exposed. The label is a composite of a plurality of layers adapted to be adhered at its underside to a product container. The label includes a printable surface layer, a longitudinal wicking strip that is adhered underneath the surface layer substantially at the opposite extremities only of the wicking strip, and a lower substrate layer forming an envelope with the said surface layer. A heat-fusible substance, which melts and flows above a pre-determined temperature, is applied on the surface of the wicking strip contiguous to at least one of the ends of the wicking member. When the heat-fusible substance is exposed to a temperature above the pre-determined temperature, the heat-fusible substance flows along the length of the wicking member.

The label has a printable surface layer and is sealed at its peripheral edge to the peripheral edge of the substrate layer. These layers encapsulate the wicking member and the heat-fusible substance. The surface layer is provided with a sight window at an intermediate location over the wicking member through which the progress of flow on the wicking member is observed. Examines labels for how long a sensitive product has been exposed to temperatures above its standard and tolerance.

In the U.S. Pat. No. 6,544,925B1 patent with the title of “Activatable time-temperature indicator system” an activatable time-temperature indicator system has been claimed to be useful in tracking the thermal exposure history of a temperature-sensitive perishable product and providing a visually distinct signal, such as a change in color density, at the expiration of a predetermined time-temperature integral comprises a first element, such as a direct thermal printing label (11), comprising a composition (13) having at least a first co-reactant of a color-forming reaction.

A second, activator element, such as an adhesive tab (21) capable of being affixed to the label element, comprises an activator component (25), such as a second co-reactant of the color-forming reaction or a solubilizing agent for prompting the interaction of the co-reactants of the label composition. The activator tab element is affixed to the label composition to activate for reaction at ambient conditions the normally high-temperature color-forming composition (13) of the label at about the same time as the label is affixed to the perishable product. Such an ability to affect the initiation of the indicator reaction at a given time eliminates any unknown, premature color formation in the indicator system to thereby ensure a true time-temperature history of the perishable product. Temperature-time-activated detectors are used. The material of the compounds used is a dye-changing compound that responds to temperature changes such as 3-pyrolidino-6-methyl-7-anilinofluoran, acid-forming reactants such as p-benzyl hydroxybenzoate, bisphenol A, phenolic density products, low-melting organic acids or esters, and cellulosic and monopolymer adhesives such as vinyl acetate, alcohols, or pyrrolidones, and acrylates or acrylamides have been used, and it has been claimed that when detected in disproportionately temperature conditions, these detectors can quantify changes by changing their color.

In U.S. Pat. No. 9,546,911B2, with the title of “Time-temperature indicator comprising a side chain crystalline polymer” a temperature-activatable time-temperature indicator has been claimed that can be used to monitor the historical exposure of a host product to ambient temperatures includes an optically readable, thermally sensitive indicator element.

The indicator element can be inactive below a base temperature and is intrinsically thermally responsive at or above an activation temperature which is equal to or greater than the base temperature. The indicator can record cumulative ambient temperature exposure above the activation temperature irreversibly with respect to time. The indicator element can include a synthetic polymeric material, and optionally, a dye.

A side-chain crystallizable polymer such as poly (hexadecylmethacrylate), that is solid below the base temperature and is a viscous liquid above the activation temperature can be employed. Intense indicator element colors can be obtained using an appropriate dye or dyes. Various structural configurations of indicators are described and illustrated. Detectors based on lateral branched crystal polymers such as polyhexadecyl methacrylate have been used to monitor the stability of temperature-sensitive products.

The functional basis of the detectors used in this claim is the use of various synthetic polymers including poly (alkyl methacrylate), one poly (tetradicylacrylate), one poly (hexadecyl methacrylate), one poly (octadecyl methacrylate), one poly (alkyl acrylate), one Poly (hexadecylacrylate), a poly (dodecyl acrylate), a copolymer of hexadacyl methacrylate, and a copolymer of tetradecylacrylate and octadecylacrylate, a copolymer of hexadecylmethacrylate and octadecylmethacrylate are used that adapt to viscous changes.

The US20190056365A1 patent with the title of “Time-temperature indicator” is generally in the field of measuring and indicating techniques and relates to a time-temperature indicator and methods of manufacturing and use thereof.

More specifically, the time-temperature indicator comprises a time-temperature indicator comprising at least one metal layer or metal-containing layer and in direct contact with the metal layer or to the metal-containing layer at least one doped polymer layer, wherein the dopant is an acid, a base, or a salt or a photo-latent acid or a photo-latent base which dopant is added to the polymer, and/or at least one polymer layer wherein a polymer is functionalized with acidic or latent acidic or basic or latent basic groups; or a time-temperature indicator comprising at least one polymer layer containing metal particles and a photo-latent acid or a photo-latent base, or at least one polymer layer containing metal particles wherein the polymer is functionalized with latent acidic or latent basic groups.

In one embodiment of the patent with the title of “A time-temperature indicator (TTI) system for temperature-sensitive products and methods for manufacturing thereof.”, an activatable TTI system is provided, comprising a multilayer assembly made of at least three functional membrane layers comprising a variable-thickness layer, a time-dependent permeability membrane layer and an indicator layer, where said variable-thickness layer has a variable thickness along the surface in contact with incoming air; said time-dependent permeability membrane has pores for ceasing further airflow into the system when it is saturated with water from the incoming air; and said indicator layer contains indicating dye which changes in color shading according to ambient temperature.

In another embodiment, a TTI system which is free from activation or preconditioning is provided, comprising an indicator strip, a sealing strip, a reservoir of reactant, and a driver, which are assembled on a substrate attached to or on the products. An indicating dye contained in the indicator strip is reactive to the reactant from the reservoir and the reactant is released according to the ambient temperature change over time, in order to indicate the change in ambient temperature by the change in the indicating dye color.

The US20090226948A1 patent with the title of “Enzyme-based time temperature indicator” relates to a time-temperature indicator for indicating temperature change over time, comprising an immobilized enzyme and a substrate of the enzyme, wherein the reaction of the substrate catalyzed by the enzyme produces a reaction product in a time and temperature dependent manner and wherein the formation of the reaction product can be detected by monitoring a physical characteristic of the substrate and/or the product which is linked to its concentration.

The invention further relates to a method of time temperature indication comprising the step of an enzyme-catalyzed reaction, a method of printing the enzyme-based time temperature indicator on a packaging material or a label, a spot of printing ink or printing ink concentrate comprising components of the enzyme-based time temperature indicator and a packaging material or a label comprising the enzyme-based time-temperature indicator.

### SUMMARY OF THE INVENTION

One of the most important factors that cause problems and food poisoning and pharmacological and biological side effects is not observing the consumption of the product within the time limit of the expiration date and looking at the expiration date of the product, which causes the production of toxic derivatives and compounds due to enzymatic activity of microorganisms., Cause side effects and destructive effects on human health. In the claimed invention, a temperature and time-sensitive chemozyme method is used to study the temperature and temporal changes of tissue and food spoilage and degradation of effective pharmacological components, and degradation of protein and glycoprotein structure of biological and medicinal products in proportion to temperature changes and its influence on increasing the speed and severity of damage and spoilage to the product, will predict the maximum useful and healthy life of the product.

### BRIEF DESCRIPTION OF DRAWINGS

FIG. 1: Shows an overview of the claimed smart tags. The top design shows the dimensions of the claimed labels on the tin or aluminum packaging surface of cans and cardboard packaging of other foodstuffs and the glass surfaces of pharmaceutical and biological vials and ampoules, and the bottom design shows the nitrocellulose structure of these labels. The scale of the provided figure is 1/5000000 to the real scale and the provided numbers are in millimeters

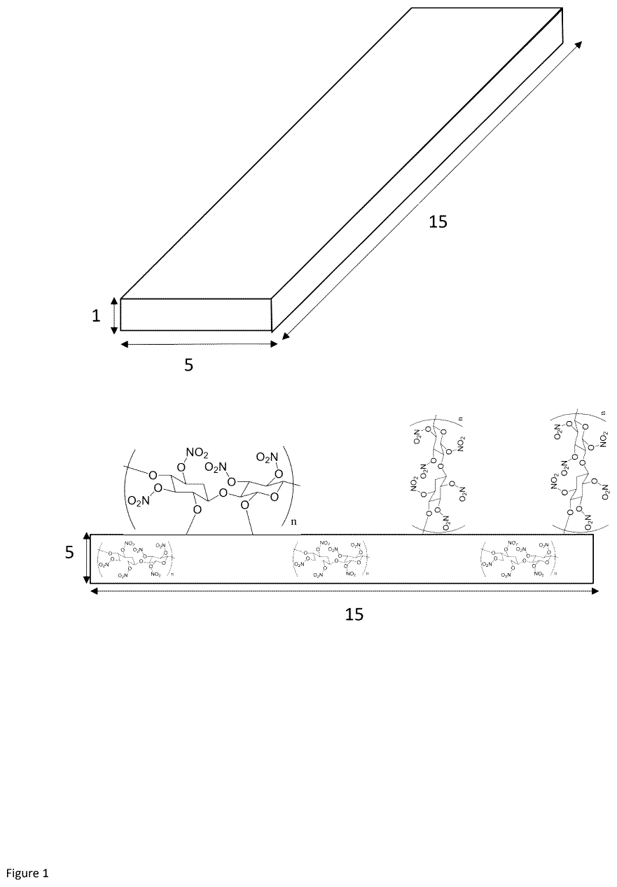

FIG. 2: The structure of type 4 protein and glycoprotein of nitrate reductase enzyme (NR) is observed and in the lower drawing, a cross-section of this laminated label is shown on the packaging surface of the product, which has a nitrocellulose structure conjugated to aqueous enzyme in aqueous medium. The scale of the provided figure is 1/5000000 to the real scale and the provided numbers are in millimeters.

FIG. 3: Illustrates the chemical structures of thermos-sensitive or temperature-sensitive polymer monomers claimed in this invention, which under the influence of radical polymerization process (1), produced lattice and porous structures of temperature-sensitive polymer (4) which with increasing temperature, reversibly and equilibrium dehydrated (2) and shrink and condense, and with decreasing temperature, the reverse process or hydration occurs (3).

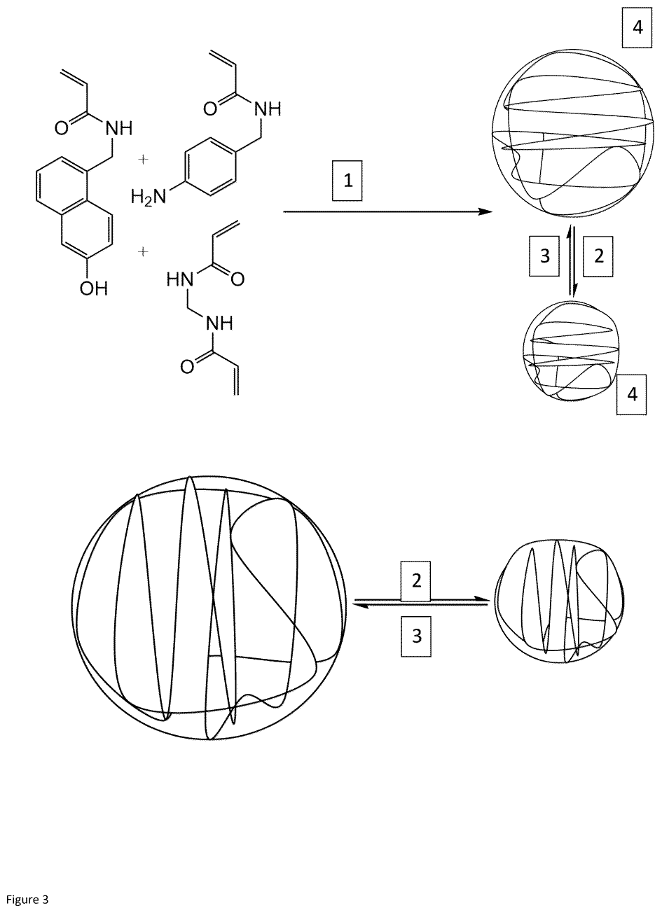

FIG. 4: Represents the chemical mechanism of polymerization of three types of monomers, depending on the w/w ratio or w/w of these three monomers and the temperature and thermos-dynamic conditions used to perform this reaction, the values of m, n, z, and w change.

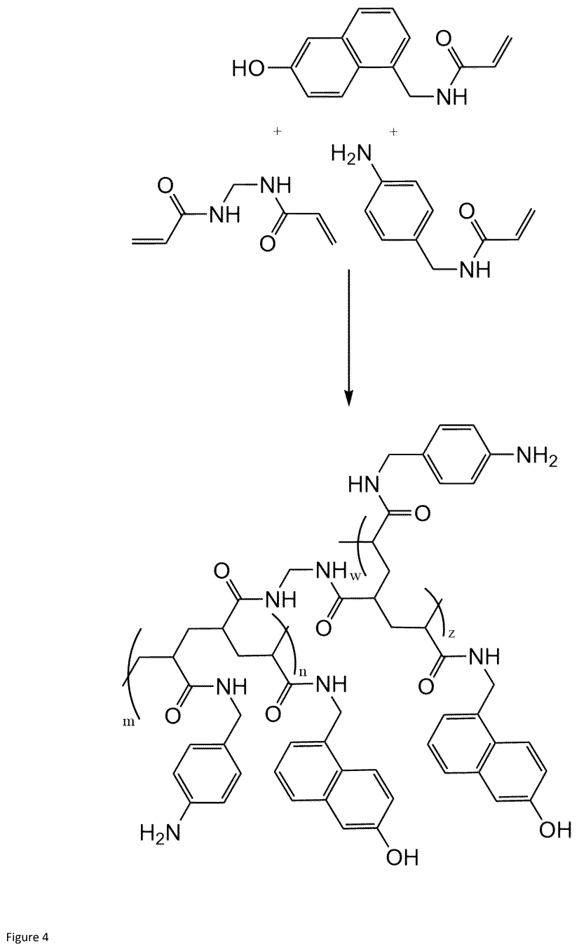

FIG. 5: Demonstrates the effect of nitrate reductase (NR) enzyme on the peripheral parts of nitrocellulose with increasing temperature and time, produced by separating nitrate substrate, cellulose, and nitrite anion. The scale of the provided figure is 1/5000000 to the real scale and the provided numbers are in millimeters.

FIG. 6: It has shown the chemical reaction of oxide and reduction of nitric acid and nitrite with 4-aminophenyl substitution and production of diazonium derivative (7) which reacts with 2-naphthol substitution in other parts of the polymer structure to form a color derivative (9) irreversibly.

### DETAILED DESCRIPTION OF THE INVENTION

After production, all food, pharmaceutical, and biological products used in the society and the health system of the country have a specific and limited time for clinical use. In fact, over time, due to the growth of opportunistic microorganisms such as fungal spores and bacterial endosperms on the one hand, and the induction of enzyme activity in the tissue of food products on the other, as well as the destructive effects of oxidizing agents in the environment such as oxygen, ultraviolet sunlight and electromagnetic radiation in the stability of drug APIs and the second, third and fourth protein structures of biological products on the other hand, by increasing the production of toxic and poisonous agents and by reducing the beneficial tissue of the product, the profitability of the product is greatly reduced and, therefore, clinically, it will be ineffective. Increasing the temperature, due to the increase in the activities of enzymes in the tissue of food products and microorganisms, has significant effects on reducing the useful life of food, pharmaceutical, and biological products.

Also note that due to the lack of an efficient system in monitoring the expiration date and useful life of products, there is a possibility of fraud and counterfeiting in the expiration date, and many problems, food poisoning, and rare side effects of pharmaceutical and biological products in PMS studies such as the production of botulism toxin in canned food and severe skin and rare systemic complications such as DRESS, SJS, TEN, anaphylactic shock and anaphylactic, result from the use of an intelligent system to detect and monitor temporal and temperature changes for ensuring the authenticity of the product seems to be very crucial.

As mentioned previously, one of the most important factors causing problems and food poisoning and pharmacological and biological side effects is the non-observance of product consumption within the expiration date of the product and looking at the expiration date of the product, which causes the production of toxic derivatives and compounds. Due to the enzymatic activity of microorganisms, it causes side effects and destructive effects on human health. In the claimed invention, it uses an intelligent method based on chemical intelligence and thermo-sensitive polymers which are acute to temperature and changes in enzymatic activity. In the latter approach, monomeric derivatives of PAM have been used to produce thermos-sensitive polymers and the enzyme nitrate reductase (NR).

The label is based on nitrocellulose on the surface of this nitrocellulose and the claimed enzyme is conjugated physically and not chemically, and moreover the claimed thermos-sensitive polymer is hydrated on the surface of this nitrocellulose layer. When food, pharmaceutical, and biological products become sensitive to temperature changes or with meta-stability conditions other than their active and consequently short half-life, it causes destruction and ineffectiveness of the effective and active component of the product, which practically complicates the clinical uses of the product.

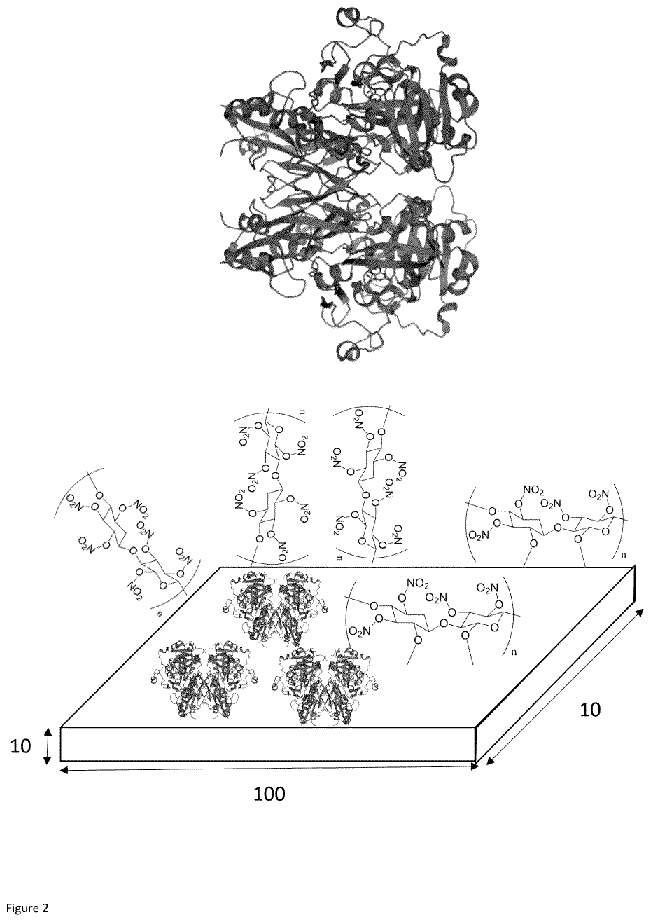

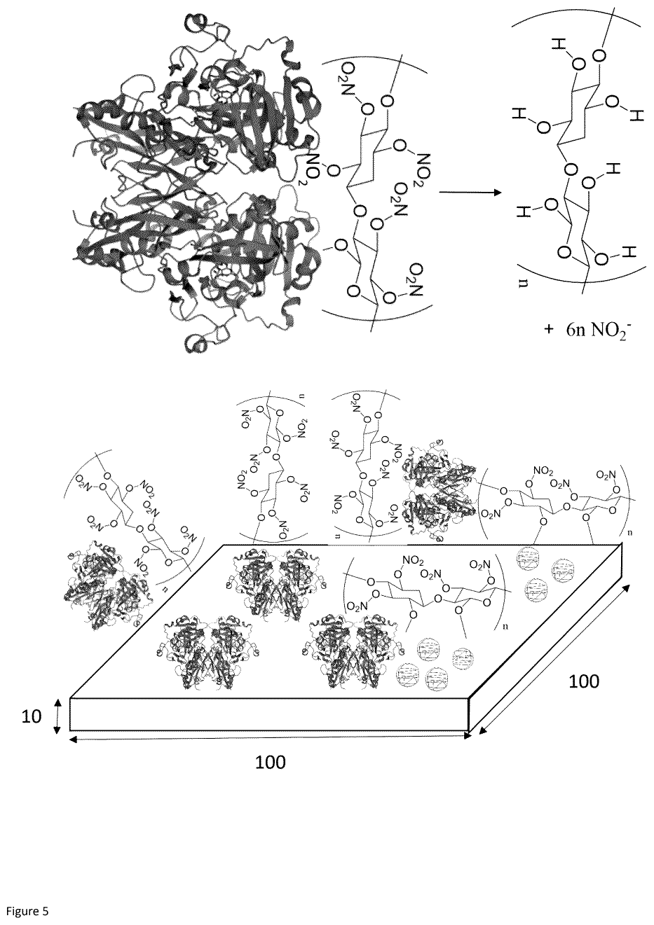

The presence of this intelligent temperature-time-sensitive expiration date monitoring system helps to adapt to changes in environmental and climatic conditions, from hot and humid to cold and dry climates. As a result, the expiration date of the product should be realistic and optimistic according to the climatic conditions. As shown in the diagram below in FIG. 5, a cross-sectional view of the claimed label is represented. On the nitrocellulose surface, the claimed enzyme is conjugated and hydrated with the claimed thermos-sensitive polymer. When the temperature rises, due to the fact that in the liquid medium, the claimed thermos-sensitive polymer has the LCST condition or lower critical solution temperature (lower critical solution temperature). Dehydrated (2) and by losing some of its water into the label space, as shown in FIG. 2 below, it becomes small unstable, and prone to chemical changes.

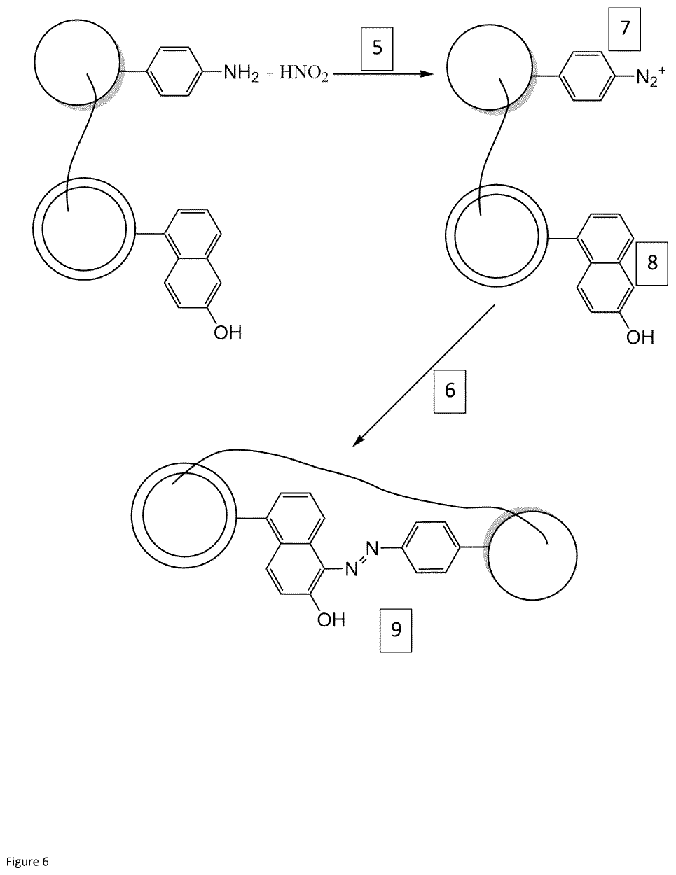

In parallel, by increasing temperature and changes in climatic conditions to hot and humid ones that increase both humidity and air temperature, the activity of the claimed enzyme increases and, consequently, the ability to hydrolyze and destroy the nitrocellulose tissue increases according to the label claimed in the current invention which is found and according to the above plan of FIG. 5, it causes the nitro extraction section connected to the end hydroxy groups and is decomposed and hydrolyzed and with the production of nitrite, the conditions for oxidation and reduction reaction (5), shown in FIG. 6.

As a result of the oxide reaction and reduction between the nitrite ion and the amine fraction of the aniline substituent, diazonium ion (7) is produced, which is exposed to the 2-naphtholium substitution (8) present in other regions of the dehydrated polymer, the nucleophilic reaction (6) and dye 2, so Aniline oil (9) is produced. This color change is irreversible and the intensity of the color can be proportional to the degree of spoilage in the tissue of the food product and degradation in the active component of the pharmaceutical-biological product.

According to the previous explanations, these claimed smart labels use a system consisting of a nitrocellulose layer, an enzyme with reductase activity, and a derivative of a PAM-based thermos-sensitive polymer.

In the claimed invention, due to the presence of two factors of thermos-sensitive polymers and enzymes, the study of the effects of temperature changes with sensitivity is much more precisely adjustable than the product.

It is sensitive to climatic conditions. In fact, in the thermo-sensitive polymers claimed in this invention, due to their sensitivity dependent on the amount of moisture and the percentage of hydration of the polymer structure, the presence of moisture can affect the protein function and enzymatic function of the claimed enzyme. The result is a more realistic approach to predicting the actual expiration dates of these products, especially pharmaceutical, and biological products that are sensitive to moisture and prone to microorganism growth.

In the claimed claim, a sensitive process with dual action properties or thermo-hydro-sensitive enzyme-polymeric dual function is used. In addition, depending on the type of food, drug, and biologic-sensitive product and according to the characteristics and standard characteristics of stability toxicity, efficacy, and health of the product in clinical use in a certain temperature-time range, it can be controlled by adjusting the percentage of monomers claimed in the radical polymerization process, and the production of desired thermo-cysts or desirable physicochemical and thermo-dynamic properties on the one hand, and calculating its concentration and coated on the nitrocellulose surface, on the other hand, it can be adjusted, measured, and monitored. the realistic expiration date of the claimed sensitive products in the climatic conditions is used, within the standard health range specified by the FDA.

To generate the claimed intelligent label in this claimed invention, it is sufficient to use the nitrocellulose layer surfaces with the claimed dimensions as the basis of the claimed label, which uses the process of physical conjugation of enzymes and polymers to coat enzymes and thermos-synthetic polymer derivatives. It is claimed that by making certain concentrations of the claimed polymer and enzyme, in an aqueous medium with pH-adjusted standard buffer in two separate vials, and uniform dispersing and dispensing of these two prepared solutions on the surface of the claimed nitrocellulose layer, drying the prepared layers at room temperature and reduced pressure decreases the surface moisture by 2-12%, depending on the type and physicochemical characteristics and temperature-time sensitivity of the product. Labels are prepared and ready to be installed on cardboard, metal, and glass surfaces and other packaging forms will have the claimed sensitive product.

Notably, the industrial application of the claimed invention, in identifying and monitoring the expiration date of food, pharmaceutical, and biologically sensitive products by embedding these nitrocellulose-based labels in the form of cardboard, aluminum-tin, and glass packaging of the products mentioned in the form of industrial products to improve QA and QC levels besides food and clinical sensitive products to increase confidence in the health and quality of production and processing of sensitive products.

1. In this invention, a design of an intelligent label has been claimed to predict, detect, and monitor the expiration date of sensitive food, pharmaceutical, and biological products to identify the realistic shelf life of various food, biological, and drug-related products and their expiration date, which includes the following sections: Nitrocellulose layer with specific dimensions as the basis of the claimed smart label Nitrate reductase enzyme, PAM monomeric derivative polymer with dual properties of temperature sensitivity and moisture sensitivity, hydro-thermo-sensitive

2. According to claim 1, a thermos-synthetic-hydro-sensitive polymer of PAM monomeric derivative is prepared from monomeric derivatives with polyacrylamide base as the preferred monomeric base with the preferential 2-naphthalene and 4-anilinelamide substitutes in the form of naphthol-polyacrylamide and aniline-polymethacrylate respectively. In addition to the use of PAM derivatives as preferred monomers, it is possible to use methacrylate monomeric derivatives and preferably methyl methacrylate with preferential substitutions of 2-naphthol in the form of naphthol-poly-methacrylate and 4-aniline in the form of aniline-polymethacrylate. Furthermore, the claimed enzyme nitrate reductase and the thermos-sensitive-hydro-sensitive polymer derived from PAM are physically conjugated and coated on the surface of the claimed nitrocellulose layers.

3. According to claim 2, the ratio of the PAM monomers used to produce the polymer can be adjusted to adjust the desired polymer properties for each product, and to adjust the amount of viscosity, ductility, polarity, temperature, UCST, or LCST thermos-dynamic behavior and other thermos-dynamic properties as one of the components of the claimed smart label, adjusted and changed in the desired direction. Similar conditions exist with the use of monomers with preferential methacrylate substitutions alongside PAM derivatives.

4. According to claim 1, the claimed enzyme has nitrate-reducing-hydrolyzing activity and converts it to the nitro in the label medium used.

5. According to claim 4, this reducing-hydrolyzing activity is temperature-dependent and has temperature-sensitive properties in the relevant enzyme.

6. According to claim 4, by using this reducing-hydrolyzing property, with increasing temperature changes and changing climatic conditions to a hot and humid condition, nitrate substitutions of nitrocellulose layers corresponding to increased enzymatic activity are converted to nitrite ions in the used label medium. As a result of this chemical reaction, the nitrocellulose layer is degraded and forms a cellulosic structure in the label medium.

7. According to claim 6, due to the structural change and deformity of the hard layer nitrocellulose with high strength quality to flexible cellulose layers, structural change in the material and the degree of flexibility of the label are created, which is one of the factors identifying and monitoring effective conditions to reduce shelf life and transition from the date of realistic expiration of the product.

8. According to claim 6, because using monomers with preferential substitution of 2-naphthol and 4-aniline in the PAM or polymethacrylate base, oxidation, and reduction reaction happens in the presence of nitrite ions obtained from the nitrocellulose degradation process, in the presence of the moisturized region of the claimed label medium and by producing diazonium derivatives as intermediate and azo color derivatives by polymer's deformation, and color change, is confirming one of the other identifying factors in monitoring the conditions affecting the reduction of shelf life and passing the realistic expiration date of the product.

9. According to claim 8, the claimed intelligent label has a dual active diagnostic and predictive behavior in the form of color change in the enzymatic structure and change in appearance and consistency in the polymer structure, in predicting, estimating, and recognizing expiration date and realistic product life which is sensitive to climate conditions.
